(19) United States
(12) Patent Application Publication
(10) Pub. No.: US 2004/0253320 A1
(43) Pub. Date: Dec. 16, 2004

(54) Nutritional supplementation for treating deficiency states in bowel disease

(75) Inventor: Pardeep Nijhawan, Thornhill (CA)

Correspondence Address: IVOR M. HUGHES, BARRISTER & SOLICITOR,, PATENT & TRADEMARK AGENTS, 175 COMMERCE VALLEY DRIVE WEST, SUITE 200, THORNHILL, ON, L3T 7P6, CA

(73) Assignee: Medical Futures Inc.

(21) Appl. No.: 10/865,986

(22) Filed: Jun. 12, 2004

(30) Foreign Application Priority Data
Jun. 13, 2003 (CA) 2,432,358

(51) Int. Cl. A61K 33/26; A61K 33/24; A61K 33/32; A61K 31/355; A61K 31/525; A61K 31/51; A61K 31/12

(52) U.S. Cl. 424/638; 424/654; 424/643; 424/646; 514/52; 514/168; 514/251; 514/276; 514/350; 514/355; 514/561; 514/690; 514/458; 514/474; 424/702; 424/667; 424/682

(57) ABSTRACT

An easily absorbed and well tolerated proportional nutritional supplementation composition and method to be used in patients suffering from deficiency states due to bowel disease that may include malabsorption and inflammatory bowel disease. It is this unique proportional formulation of antacid, calcium, vitamins, minerals and simethicone in lactose free, sorbitol free, gluten free and hypo allergen solution that is the novel invention. This novel invention allows for nutrients to be readily absorbed with little or no side effects that may be experienced when taking conventional supplements. The supplementation contains amounts of vitamins and minerals that are commonly deficient in these states including magnesium, chromium, increased iron, increased folic acid, calcium and fat soluble vitamins.

### FIELD OF THE INVENTION

The invention is to be used as a nutritional supplementation along with diet and prescribed medications for patients suffering from digestive syndromes. This includes but is not limited to inflammatory bowel disease, irritable bowel syndrome, peptic ulcer disease, undiagnosed constipation, and undiagnosed diarrhea and malabsorption syndromes. The invention includes a proportional supplement designed to decrease the number of tablets taken by patients suffering from the above conditions while maximizing absorption and minimizing side effects.

### BACKGROUND OF THE INVENTION

There are many patients who suffer from bowel disease. It is estimated that the approximately 15% of the general population at anytime may suffer from bowel symptoms. A smaller segment of the general population suffers from either Inflammatory Bowel Disease (IBD) or Malabsorption syndromes. There are a number of distinct disease states in each of the two categories. Inflammatory bowel disease includes Crohn's, Ulcerative Colitis, Collagenous Colitis and non-specific colitis. Due to the inflammation in the bowel, many patients loose important vitamins and minerals through their bowels. In addition, in those who have small bowel lesions, they may experience an inability to absorb adequate amounts of certain vitamins and minerals.

Deficiency states may be aggravated by patients who are on certain medications (e.g. sulfasalazine, methotrexate) that interfere with absorption of vitamins and minerals. As a result, many current supplementations include high doses of vitamins and minerals to over come these deficiency states. However, the high doses of vitamins and minerals result in increased intolerance and side effects.

These side effects may include bloating, belching, cramps, dyspepsia, constipation, diarrhea, increased flatulence and nausea.

These symptoms may increase the digestive complaints of patients who have food allergies or irritable bowel syndrome. This present invention addresses these complaints by creating a new proportional low side effect mixture.

Many patients who currently use supplements find that they either are unable to absorb the multivitamins or they have increased side effects from the regular use of the multivitamins. Some of these side effects result from unabsorbed nutrients given in their conventional supplement due to excess quantities beyond absorption thresholds. We all have receptors in our bowels that assist with nutrient absorption. If these receptors become saturated, they will no longer continue to absorb nutrients. Therefore, once the threshold is reached, the patients will expel most of the nutrients into their stools. It is the excess amount of nutrients that can cause a number of side effects. For example, iron is commonly prescribed in large doses by physicians even though many patients cannot absorb more than one 325mg a day. Any excess that is left in the bowels will result in constipation and cramps. By simply reducing the quantity of iron, we can still maintain maximum absorption while causing minimum side effects.

Other additives can also reduce the incidence of bowel symptoms in patients. These include the addition of antacids such as calcium and of antiflatulents such as simethicone. Simethicone has been shown to decrease gas formation and can help decrease the gas formation that may occur from certain compounds in supplements. For example, it would be advantageous to provide a supplement with a high dose of calcium in a format such as bicarbonate that may result in increase gas formation, to include simethicone to reduce gas formation and allow for decrease adverse effects. This would allow the patients to take such as proportional nutritional supplement through flare-ups of their irritable bowel or other bowel conditions. Simethicone has been commercially available in tablet and liquid form. It is also available as a pharmaceutical excipient in topical formulations. Heretofore such a supplement is unknown to the inventor.

U.S. patent 6,495,177 by deVries et al has a supplementation that contains simethicone with a formulation that is free of calcium. The present invention not only includes simethicone, but also the substantially ideal doses of minerals and vitamins required by the patients including a high dose of calcium. It is this unique formulation of calcium, vitamins, minerals and simethicone in a lactose free, sorbitol free, gluten free and allergen free solution that overcomes the lack in the prior. art and is the novel for reducing bowel side known effects with multivitamins. DeVries patent does not teach a composition according to the present invention.

Calcium provides antacid effects. This will quickly increase the pH level within the stomach resulting in a soothing affect for the patient who may be suffering from dyspepsia. It would therefore be beneficial to provide a formulation, to enable a patient to consume three tablets a day, to achieve the calcium requirement of the American College of Gastroenterology to statistically reduce the chance of formation of colon polyps.

Any supplement should also contains magnesium. Magnesium is essential for the functioning of a number of critical enzymes involved with ATP-dependent phosphorylation, protein synthesis, and carbohydrate metabolism. Magnesium is the second most common cation in the body. Magnesium deficiency can occur due to poor absorption as a result of previous surgical resections, chronic diarrhea or fistula losses. Serum levels reflect approximately 10 percent of body stores. Magnesium deficiency may result in tetany, ataxia, myoclonic jerks, coma, psychosis, cardiac dysrhthmias and hypotension.

It would therefore be advantageous to provide a supplement having sufficient magnesium in the formulation to (1) allow proper absorption of other minerals and vitamins (2) allows adequate replacement of magnesium deficiency states in bowel disease and (3) is able to counter the effects of calcium and iron on bowel movements by decreasing the incidence of constipation through its laxative properties. By controlling the amount in the formulation can prevent diarrhea.

Chromium is essential in patients with IBD. Chromium is needed for protein, carbohydrate and lipid metabolism and is also crucial for the synthesis of glucose tolerance factor, a cofactor in insulin action. True chromium deficiency is manifested by glucose intolerance. Chromium requirements are 50-100 mg/day.

In malabsorption states, patients suffer from an inability of the bowel to absorb nutrients. There are numerous factors for this including an inability to break down food, decreased mucosal transportation activity, lack of enzymes, and large amounts of exudates. Some conditions that can lead to this include lymphoma, celiac disease, abetalipoproteinemia and short bowel syndrome.

Additionally, deficiency states can result from insufficient intake, increased gastrointestinal losses (such as fistula fluids, diarrhea and intestinal inflammation), and increased nutritional requirements (such as infection).

One major reason that IBD and malabsorption syndrome patients have insufficient intake is due to a loss of appetite. Many of these patients suffer from chronic abdominal pain and diarrhea resulting in a loss of appetite. Furthermore, food restrictions also decrease the opportunity for a balanced diet.

Crohn's disease patients may experience fat malabsorption due to terminal ileum resections or terminal ileitis. These patients are susceptible to malabsorption due to prescription medications such as prednisone, which results in calcium loss and inhibition of calcium absorption. Others medications such as sulfasalazine cause folate deficiency and cholestyramine results in loss of fat-soluble vitamins.

Vitamin B 12 deficiency is common in inflammatory bowel disease. Fat soluble vitamins (A, D, E and K) absorption is impaired in patients with small intestinal disease or in those who have had small intestinal resections. Low levels of vitamin D may result in bone loss in some patients. A low levels of Vitamin A may result in impaired night vision. Vitamin K deficiency may result in increased bruising or, rarely, bleeding.

Vitamin C, which is not a fat soluble vitamin, can also be deficient in states of inflammatory bowel and malabsorption syndromes. High doses of vitamin C in supplements may not be absorbed by the bowel. This could result in patients loosing a large amount of vitamin C in their stool. It would therefore be advantageous to the patient to be able to reach the maximum absorption level that is possible under substantially ideal conditions.

Iron deficiency is common in IBD due to the trace amounts of blood lost through the inflamed and ulcerated bowel. Iron, carried in red blood cells helps with oxygenation. Therefore, it is important to replace lost iron with supplementation in affected patients.

However, patients who take large doses of iron have interactions with other drugs such as levothyroxine. It would therefore be advantageous to the patient to include the amount of iron that should provide the highest percentage of absorption with the least amount of interaction.

Folic acid is required to help create and mature red blood cells and prevent certain disease states. This includes things such as anemia and in women who are of child bearing age neural tube defects in the newborn. In addition, certain patients are prescribed medications, such as sulfasalazine which can lead to folate deficiency, requiring the need for folic acid supplementation. Several papers have shown a correlation with increased colon polyp formation in patients who are folate deficient. Any supplement should have enough folate to correct the deficiency state and potentially prevent the formation of polyps.

Copper losses occur in patients with diarrhea fistula drainage and increased ileostomy losses. In addition, approximately 40% of patients with Crohn's disease have low levels of plasma zinc. Zinc is important in wound healing cell-mediated immunity and normal growth in children. Once again it would be advantageous to meet the required daily values of these two minerals in a supplement.

Patients with inflammatory bowel disease and malabsorption commonly suffer from metabolic bone disease. Calcium is the main component of bone. Calcium absorption also helps prevent oxalate absorption, which is a known cause of oxalate stones in patients with Crohn's disease. By supplementing the calcium previously this effectively reduces this condition from occurring.

Other patients suffer from lactose intolerance. This is common in patients older than 30 years of age. A formulation that is lactose free will allow for easy absorption without causing bloating and malabsorption symptoms.

In addition, all compounds in a supplement should be gluten free. Gluten is a protein found in certain food compounds, which is poorly tolerated by patients with Celiac disease. The present formulation should be gluten free and should help prevent malabsorption in these patients while providing them with the vitamin and mineral supplementation that is required.

Most vitamins are created with “filler compounds”. In some cases, these compounds are derived from proteins that cause allergic reactions in patients who suffer from food allergies. The most common groups of food allergies include milk, egg, soy, wheat, seafood and nuts due to certain protein products. Any supplement should not include substances that would trigger such a response. This hypo allergen nutrient mixture with minimal side effects is contemplated with this invention.

Several patents have been disclosed nutritional supplementation. However, these represent different methodology or are of different composition. U.S. Pat. No 4,617,317 to Bennet disclosed administration of vitamin K antagonists. Their formulation differs significantly to the present invention. The second U.S. Pat No. 5,780,451 to DiMichele et al discussed the composition of a mixture of an oil blend and a source of indigestible carbohydrates which are readily converted to short chain fatty acids. This is intended for enteral nutrition and not for oral delivery mechanisms.

U.S. Pat. No. 6,214,373 to Snowden disclosed an oral nutritional composition and method for inflammatory bowel disease. Snowden's composition is free of magnesium, an essential component of any nutritional supplement. It is not formulated to cause a decrease in side effects such as cramping and bloating. It does not contain any simethicone to assist with gas formation. In addition, other differences include the lack of chromium, the lower quantities of calcium, iron and folate found in Snowden's composition. Therefore, this patent does not teach a composition according to the present disclosure.

While numerous multi-vitamin and mineral supplements are in the art, none of these prior publications or supplements have been formulated in the correct proportions and with additives (i.e. the antacid and antiflatulents) to allow for maximum nutrient absorption with the fewest side effects.

### SUMMARY OF THE INVENTION

According to a primary of the aspect of the invention there is provided all of the embodiments found in detail in the attached claim set.

The invention is a nutritional composition of minerals and vitamins that are commonly found to be deficient in patients who are suffering from bowel diseases such as inflammatory bowel disease and malabsorption syndromes.

The nutritional supplementation of vitamins and minerals contains high enough amounts of calcium to have an antacid effect. The nutritional supplementation of vitamins and minerals has a pharmaceutically acceptable calcium compound selected from the group consisting of calcium carbonate, calcium sulphate and mixtures thereof.

The nutritional supplementation of vitamins and minerals contains simethicone to provide an antiflatulents effect.

The nutritional supplementation of vitamins and minerals will have a composition that is hypo allergic in nature.

The nutritional supplementation of vitamins and minerals provides individuals with sufficient amounts of essential minerals and vitamins. These include the fat soluble vitamins (A, D, E and K).

The nutritional supplementation of vitamins and minerals has increased levels of chromium to be provided to the individual by mouth.

The nutritional supplementation of vitamins and minerals has pharmaceutically-acceptable magnesium present preferably as magnesium stearate.

The nutritional supplementation of vitamins and minerals has iron present preferably as ferrous fumarate in a concentration of at least 20 mg.

The nutritional supplementation of vitamins and minerals has at least a concentration of 0.4 mg of folic acid.

The nutritional supplementation of vitamins and minerals has at least a concentration of 5 mcg chromium.

The nutritional supplementation of vitamins and minerals, wherein the composition further comprises a pharmaceutically acceptable excipient selected from any of the group consisting of: hydroxypropylcellulose and starch.

The nutritional supplementation of vitamins and minerals, wherein its composition is in the form of a tablet, caplet, capsule, dermal patch or a suspension.

### DETAILED DESCRIPTION OF THE INVENTION

This new invention is a nutritional supplementation of vitamins and minerals that are beneficial for patients who suffer deficiency states due bowel conditions. This nutritional supplementation would be administered by mouth twice a day. It is unique due to the composition of high concentrations of certain minerals and vitamins, especially magnesium, calcium, copper, chromium, iron and folate without causing increased side effects.

The composition of this nutritional supplementation includes vitamins and minerals in the range of amounts shown below;

| | | |
|---|---|---|
| Vitamin A | 2500 to 5000 | IU |
| Beta Carotene | 2500 | IU |
| Vitamin D3 (cholecalciferol) | 200 to 400 | IU |
| Vitamin B1 | 1.0 to 6.0 | mg |
| Vitamin B2 | 1.0 to 6.0 | mg |
| Vitamin B6 | 1.0 to 6.0 | mg |
| Vitamin B12 | 100 to 200 | mcg |
| Vitamin K | 0 to 60 | mcg |
| Niacinamide | 10 to 20 | mg |
| Vitamin C | 30 to 60 | mg |
| Pantothenic Acid | 8 to 10 | mg |
| Iron | 20 to 60 | mg |
| Calcium | 400 to 650 | mg |
| Zinc | 5 to 15 | mg |
| Folic Acid | 0.4 to 1.0 | mg |
| Vitamin E | 30 to 60 | IU |
| Selenium | 10 to 50 | mcg |
| Iodine | 40 to 80 | mcg |
| Copper | 0 to 2 | mg |
| Chromium | 5 to 20 | mcg |
| Magnesium | 5 to 30 | mcg |

Many patients who currently use supplements find that they either are unable to absorb the multivitamins or they have increased side effects from the regular use of the multivitamins. Some of these side effects result from unabsorbed nutrients given in their conventional supplement due to excess quantities beyond absorption thresholds. We all have receptors in our bowels that assist with nutrient absorption. If these receptors become saturated, they will no longer continue to absorb nutrients. Therefore, once the threshold is reached, the patients will expel most of the nutrients into their stools. It is the excess amount of nutrients that can cause a number of side effects. Having the right amount of nutrient is important from both an absorption and from an interaction point of view.

Other additives can also reduce the incidence of bowel symptoms in patients. These include the addition of antacids such as calcium and of antiflatulents such as simethicone. Simethicone has been shown to decrease gas formation and can help fight the gas formation that may occur from certain compounds in this supplementation. For example, the supplement will have a high dose of calcium in a format such as bicarbonate that may result in increase gas formation. By adding simethicone, we are able to reduce this formation and allow for decrease adverse events from our preparation. This allows the patients to take this proportional nutritional supplement through flare-ups of their irritable bowel or other bowel conditions.

It is essential that the composition of this nutritional supplementation include magnesium. Patients who are deficient in magnesium can suffer potentially fatal outcomes. The specific amount of magnesium in the formulation allows us to (1) allow proper absorption of other minerals and vitamins (2) allows adequate replacement of magnesium deficiency states in bowel disease and (3) is able to counter the effects of calcium and iron on bowel movements by decreasing the incidence of constipation through its laxative properties. The amount of magnesium in the formulation can prevent diarrhea, cardiac dysrhthmias, tetany, ataxia, myoclonic jerks, coma, psychosis, and hypotension.

It is essential that the composition of this nutritional supplementation include chromium. Patients who are deficient in chromium can suffer from defective metabolism of protein, carbohydrate and lipids.

The composition of this nutritional supplementation includes high amount of copper in order to replace losses that may occur from fistula drainage.

The composition of this nutritional supplementation also includes high amounts of iron and folic acid.

The composition of this nutritional supplementation also includes high amount of calcium to prevent metabolic bone disease. This calcium is in a form that is easily absorbed from the bowels into serum.

The preferred formulation of the invention is shown below:

| | | |
|---|---|---|
| Vitamin A | 2500 | IU |
| Beta Carotene | 2500 | IU |
| Vitamin D3 (cholecalciferol) | 400 | IU |
| Vitamin B1 | 1.5 | mg |
| Vitamin B2 | 1.7 | mg |
| Vitamin B6 | 2 | mg |
| Vitamin B12 | 100 | mcg |
| Niacinamide | 20 | mg |
| Vitamin C | 60 | mg |
| Pantothenic Acid | 10 | mg |
| Iron | 27 | mg |
| Calcium | 450 | mg |
| Zinc | 15 | mg |
| Folic Acid | 0.4 | mg |
| Vitamin E | 30 | IU |
| Selenium | 20 | mcg |
| Iodine | 60 | mcg |
| Vitamin K | 0 to 15 | mg |
| Copper | 2 | mg |
| Chromium | 20 | mcg |
| Magnesium | 10 | mcg |

Calcium in the doses provided for in this mixture, when ingested one tablet twice a day provides antacid effects. This will quickly increase the pH level within the stomach resulting in a soothing affect for the patient who may be suffering from dyspepsia.

Our formulation is allergen free for example egg products, wheat (gluten) and dairy (lactose) free which will allow for easy absorption without causing bloating and malabsorption symptoms.

In addition, all compounds in our formulation are gluten free. Gluten is a protein found in certain food compounds that result in patients who suffer from Celiac disease to experience a malabsorption syndrome. Our formulation is gluten free and should help prevent malabsorption in this patient population while providing them with vitamin and mineral supplementation that is typically required by these patients.

Most vitamins are created with filler compounds. In some cases, these compounds are derived from proteins that cause allergic-reactions in patients who suffer from food allergies. The most common groups of food allergies include milk, egg, soy, wheat, seafood and nuts due to certain protein products. In our novel formulation, we have included no substances that would trigger such a response. This hypo allergen nutrient mixture combination with minimal side effects is a novel invention.

The new composition is unique and not been described with all of its individual features in one effective formulation in the prior supplements.

| US Patent Documents | | |
|---|---|---|
| 4617317 | October 1986 | Bennet |
| 5780451 | July 1998 | DeMichele et al |
| 6214373 | April 2001 | Snowden |
| 6495177 | December 2002 | deVries et al |

The present invention is not to be limited in scope by the embodiments disclosed herein in the tables, which are intended as single illustrations of one aspect of the invention, and any which are functionally equivalent are within the scope of the invention. Accordingly, the claims and specification should not be construed to unduly narrow the full scope of protection to which the present invention is entitled.

The embodiments of the invention in which an exclusive property or privilege is claimed are as follows:

1. A proportional therapeutically effective nutritional supplement comprising a mixture of vitamins and minerals to be administered to patients suffering from bowel disease formulated in a hypo allergen form providing improved absorption and tolerance with less side effects than conventional supplements wherein said supplement is formulated to include the levels of vitamins and minerals indicated to ensure that the nutrient receptors in the bowel of the patient are not saturated so as to prevent absorption of said nutrients and the unwanted side effects in the bowel of receptor saturation.

| | | |
|---|---|---|
| Vitamin A | 2500 to 5000 | IU |
| Beta Carotene | 2500 | IU |
| Vitamin D3 (cholecalciferol) | 200 to 400 | IU |
| Vitamin B1 | 1.0 to 6.0 | mg |
| Vitamin B2 | 1.0 to 6.0 | mg |
| Vitamin B6 | 1.0 to 6.0 | mg |
| Vitamin B12 | 100 to 200 | mcg |
| Vitamin K | 0 to 60 | mcg |
| Niacinamide | 10 to 20 | mg |
| Vitamin C | 30 to 60 | mg |
| Pantothenic Acid | 8 to 10 | mg |
| Iron | 20 to 60 | mg |
| Calcium | 400 to 650 | mg |
| Zinc | 5 to 15 | mg |
| Folic Acid | 0.4 to 1.0 | mg |
| Vitamin E | 30 to 60 | IU |
| Selenium | 10 to 50 | mcg |
| Iodine | 40 to 80 | mcg |
| Copper | 0 to 2 | mg |
| Chromium | 5 to 20 | mcg |
| Magnesium | 5 to 30 | mcg |

2. The nutritional supplement according to claim 1 further comprising simethicone.

3. The nutritional supplement according to claim 1 or 2 wherein said supplement is free of allergens such as but not limited to lactose, gluten, egg products, soy products, seafood, nuts or sorbitol.

4. The nutritional supplement according to claim 1, wherein the therapeutically effective calcium component is selected from the group consisting of calcium carbonate, calcium sulphate and mixtures thereof.

5. The nutritional supplement of claim 1 or 2, wherein the therapeutically effective magnesium is present as magnesium stearate.

6. The nutritional supplement of claim 1 or 2, wherein iron is present as ferrous fumarate in a concentration of at least 20 mg.

7. The proportional nutritional supplement of claim 1 or 2, wherein the composition has at least 2000 IU of Beta Carotene.

8. The nutritional supplement of claim 1 or 2, wherein the composition has at least 0.4 mg of folic acid.

9. The nutritional supplement of claim 1 or 2, wherein the composition has at least 5 mcg of chromium.

10. The nutritional supplementation of claim 1, wherein the composition further comprises a pharmaceutically acceptable excipient selected from any of the group consisting of: hydroxypropyl cellulose and starch.

11. The nutritional supplementation of claim 8, wherein the composition is in the form of a tablet, caplet, capsule, dermal patch or a suspension.

12. The nutritional supplementation of claim 1, wherein the composition has no allergen particles that may cause typical seafood, nuts or protein food allergies.

13. The nutritional supplement of claim 1, wherein the mixture has substantially the following amounts of vitamins and minerals in a hypo allergen formulation which allows for maximum absorption and minimal side effects:

| | | |
|---|---|---|
| Vitamin A | 2500 | IU |
| Beta Carotene | 2500 | IU |
| Vitamin D3 (cholecalciferol) | 400 | IU |
| Vitamin B1 | 1.5 | mg |
| Vitamin B2 | 1.7 | mg |
| Vitamin B6 | 2 | mg |
| Vitamin B12 | 100 | mcg |
| Niacinamide | 20 | mg |
| Vitamin C | 60 | mg |
| Pantothenic Acid | 10 | mg |
| Iron | 27 | mg |
| Calcium | 450 | mg |
| Zinc | 15 | mg |
| Folic Acid | 0.4 | mg |
| Vitamin E | 30 | IU |
| Selenium | 20 | mcg |
| Iodine | 60 | mcg |
| Vitamin K | 0 to 15 | mg |
| Copper | 2 | mg |
| Chromium | 20 | mcg |
| Magnesium | 10 | mcg |

14. A method of enhancing the health of a patient who is suffering from conditions that affect the bowel such as inflammatory bowel disease or malabsorption syndromes comprising administering to said patient a therapeutically effective proportional nutritional supplement of vitamins and minerals in a hypo allergen form by mouth, intravenous, or transdermally.

| | | |
|---|---|---|
| Vitamin A | 2500 to 5000 | IU |
| Beta Carotene | 2500 | IU |
| Vitamin D3 (cholecalciferol) | 200 to 400 | IU |
| Vitamin B1 | 1.0 to 6.0 | mg |
| Vitamin B2 | 1.0 to 6.0 | mg |
| Vitamin B6 | 1.0 to 6.0 | mg |
| Vitamin B12 | 100 to 200 | mcg |
| Vitamin K | 0 to 60 | mcg |
| Niacinamide | 10 to 20 | mg |
| Vitamin C | 30 to 60 | mg |
| Pantothenic Acid | 8 to 10 | mg |
| Iron | 20 to 60 | mg |
| Calcium | 400 to 650 | mg |
| Zinc | 5 to 15 | mg |
| Folic Acid | 0.4 to 1.0 | mg |
| Vitamin E | 30 to 60 | IU |
| Selenium | 10 to 50 | mcg |
| Iodine | 40 to 80 | mcg |
| Copper | 0 to 2 | mg |
| Chromium | 5 to 20 | mcg |
| Magnesium | 5 to 30 | mcg |

wherein said supplement is formulated to include the levels of vitamins and minerals indicated to ensure that the nutrient receptors in the bowel of the patient are not saturated so as to prevent absorption of said nutrients and the unwanted side effects in the bowel of receptor saturation.

15. The method of claim 14 wherein said supplement contains simethicone.

16. The method of claim 14 or claim 15 wherein said supplement is free of lactose, gluten and sorbitol.

17. The methods according to claim 14, wherein the therapeutically effective calcium component is selected from the group consisting of calcium carbonate, calcium sulphate and mixtures thereof.

18. The method according to claim 14 or 15, wherein therapeutically effective magnesium is present in the supplement as magnesium stearate.

19. The method according to claim 14 or 15, wherein iron is present in the supplement as ferrous fumarate in a concentration of at least 20 mg.

20. The method according to claim 14 or 15, wherein the supplement has at least 2000 IU Beta Carotene.

21. The method according to claim 14 or 15, wherein the supplement has at least 0.4 mg of folic acid.

22. The method according to claim 14 or claim 15, wherein the supplement has at least 5 mcg of chromium.

23. The method according to claim 14, wherein the supplement further comprises a pharmaceutically acceptable excipient selected from any of the group consisting of: hydroxypropyl cellulose and starch.

24. The method according to claim 14, wherein the supplement is in the form of a tablet, caplet, capsule, dermal patch or a suspension.

25. The method according to claim 14, wherein the supplement contains no allergen particles that may cause typical seafood, nuts or protein food allergies

26. The method according to claim 14, wherein the supplement has substantially the following amounts of vitamins and minerals in a hypo allergen form which allows for maximum absorption and minimum side effects:

| | | |
|---|---|---|
| Vitamin A | 2500 | IU |
| Beta Carotene | 2500 | IU |
| Vitamin D3 (cholecalciferol) | 400 | IU |
| Vitamin B1 | 1.5 | mg |
| Vitamin B2 | 1.7 | mg |
| Vitamin B6 | 2 | mg |
| Vitamin B12 | 100 | mcg |
| Niacinamide | 20 | mg |
| Vitamin C | 60 | mg |
| Pantothenic Acid | 10 | mg |
| Iron | 27 | mg |
| Calcium | 450 | mg |
| Zinc | 15 | mg |
| Folic Acid | 0.4 | mg |
| Vitamin E | 30 | IU |
| Selenium | 20 | mcg |
| Iodine | 60 | mcg |
| Vitamin K | 0 to 15 | mg |
| Copper | 2 | mg |
| Chromium | 20 | mcg |
| Magnesium | 10 | mcg |

27. The method according to claim 14, wherein said supplement is administered once, twice or three times daily.
